(19) United States
(12) Patent Application Publication
(10) Pub. No.: US 2021/0163575 A1
(43) Pub. Date: Jun. 3, 2021

(54) FACTOR VIII MUTANT EXPRESSION VECTOR WITH ENHANCED PROTEIN EXPRESSION

(71) Applicants: G&P BIOSCIENCE CO., LTD., Gyeonggi-do (KR); REYON PHARMACEUTICAL CO., LTD., Seoul (KR)

(72) Inventors: Seong-Hyun HO, Seoul (KR); Su Jin PARK, Gyeonggi-do (KR)

(73) Assignees: G&P BIOSCIENCE CO., LTD., Gyeonggi-do (KR); REYON PHARMACEUTICAL CO., LTD., Seoul (KR)

(21) Appl. No.: 16/770,678

(22) PCT Filed: Dec. 5, 2018
(86) PCT No.: PCT/KR2018/015333

§ 371 Date: Jun. 8, 2020

(30) Foreign Application Priority Data
Dec. 7, 2017 (KR) 10-2017-0167405

(51) Int. Cl. C07K 14/755; A61K 48/00; C12N 15/85

(52) U.S. Cl. CPC C07K 14/755; A61K 38/00; C12N 15/85; A61K 48/00

(57) ABSTRACT

The present disclosure relates to an expression vector that carries a polynucleotide coding for a coagulation factor VIII mutant and has increased protein expression and a pharmaceutical composition containing the expression vector for preventing or treating hemorrhagic disease or hemorrhage.

The factor VIII mutant of the present disclosure is derived by deleting a part of the B-domain (residues 784-1667) and a part of the a3 region (residues 1668-1671) in factor VIII and, as a result, an expression vector carrying a polynucleotide coding for the factor VIII mutant has remarkably increased protein expression.

### TECHNICAL FIELD

The present disclosure relates to an expression vector that includes a polynucleotide encoding a coagulation factor VIII mutant and has increased protein expression and a pharmaceutical composition containing the expression vector for preventing or treating hemorrhagic disease or hemorrhage.

### BACKGROUND ART

Hemophilia A (HA) is one of severe genetic disorders. Hemophilia is a disorder related with a gene on X-chromosome and occurs in 1 out of 5000 males. It is caused by the variation of the plasma glycoprotein factor VIII (FVIII) which is an important component during blood coagulation. The factor VIII (hereinafter, FVIII) encodes 2351 amino acids and has six domains (A1-A2-B-A3-C1-C2). A heterodimer is formed by a heavy chain consisting of the A1, A2 and B domains and a light chain consisting of the A3, C1 and C2 domains.

Prior to the 1980s, patients with hemophilia received factor VIII extracted from the plasma of other people for treatment. However, this method had severe problems such as viral infection. Since the 1980s, full-length factor VIII produced in CHO cells, etc. through recombinant protein technology has been used in order to solve this problem. Later, as it is known that B domain-deleted factor VIII (F8-BDD) can be produced with high protein productivity with no difference in activity, recombinant proteins based on two-chain F8-BDD, which express a heavy chain and a light chain, respectively, have been developed and used. A recently developed truncated single-chain F8-BDD recombinant protein was proven to show 2- to 4-fold increased AUC as compared to the two-chain F8-BDD due to excellent in-vivo stability.

Although the factor VIII recombinant protein exhibits superior therapeutic effect, its concentration in the body should be consistently maintained at 5% or higher in order to prevent internal hemorrhage in patients with hemophilia. For this, the protein should be administered every day or 2-3 times a week. To overcome this disadvantage, a recombinant virus-based gene therapy agent was developed in the early 2000s. The recombinant virus-based gene therapy agent uses B-domain-deleted factor VIII (hereinafter, ‘F8-BDD’) with a size of smaller than 4.4 kb, rather than full-length factor VIII with a size of 7 kb or larger. In particular, most of therapeutic agents use the single-chain F8-BDD gene rather than the two-chain F8-BDD gene.

The inventors of the present disclosure have made efforts to develop a factor VIII gene therapy agent with further improved stability. In doing so, they have developed a single-chain F8-BDD mutant which exhibits improved stability and increased protein expression in eukaryotic cells as compared to the single-chain F8-BDD gene, and an expression vector expressing the same. In addition, they have identified that the expression and stability can be further improved by using an endogenous promoter such as elongation factor-1 alpha (EF-1a) and codon-optimizing (CO) the single-chain F8-BDD mutant, and have completed the present disclosure.

### REFERENCES OF RELATED ART

### Patent Documents

(Patent document 001) KR 10-1542752 B.

### DISCLOSURE

### Technical Problem

The present disclosure is directed to providing a factor VIII mutant expression vector with increased protein expression.

The present disclosure is also directed to providing a pharmaceutical composition for preventing or treating hemorrhagic disease or hemorrhage, which contains the expression vector.

### Technical Solution

The present disclosure provides a factor VIII mutant expression vector including a single-chain polynucleotide encoding a factor VIII mutant wherein amino acids Asp784 to Arg1671 are deleted from factor VIII represented by SEQ ID NO 1.

In an exemplary embodiment of the present disclosure, the factor VIII mutant may have an amino acid sequence represented by SEQ ID NO 3.

In an exemplary embodiment of the present disclosure, the vector may be selected from a group consisting of pCDNA3.1, pGP and pEF.

In an exemplary embodiment of the present disclosure, the expression vector may have increased protein expression as compared to an expression vector including a polynucleotide encoding factor VIII represented by SEQ ID NO 1, B-domain-deleted factor VIII or single-chain factor VIII.

In addition, the present disclosure provides an expression system for expressing a factor VIII mutant, which includes the expression vector.

In addition, the present disclosure provides a pharmaceutical composition for preventing or treating hemorrhagic disease or hemorrhage, which contains the expression vector.

In an exemplary embodiment of the present disclosure, the hemorrhagic disease may be hemophilia A, hemophilia caused or complicated by an inhibitory antibody against factor VIII or factor Villa or hemophilia B.

In an exemplary embodiment of the present disclosure, the hemorrhagic disease may be one selected from a group consisting of neonatal coagulopathy, severe liver disease, thrombocytopenia, congenital deficiency of factor V, VII, X or XI, and von Willebrand disease with inhibitors against von Willebrand factor.

In an exemplary embodiment of the present disclosure, the hemorrhage may be caused by blood loss associated with a high-risk surgical procedure, traumatic blood loss, bone marrow transplantation or cerebral hemorrhage.

In an exemplary embodiment of the present disclosure, the pharmaceutical composition may be for gene therapy.

### Advantageous Effects

An expression vector including a polynucleotide encoding a factor VIII mutant of the present disclosure, with a part of the B-domain (residues 784-1667) and a part of the a3 region (residues 1668-1671) in factor VIII deleted, has remarkably increased protein expression.

### BRIEF DESCRIPTION OF DRAWINGS

FIG. 1 schematically illustrates a process of designing a factor VIII mutant according to an exemplary embodiment of the present disclosure.

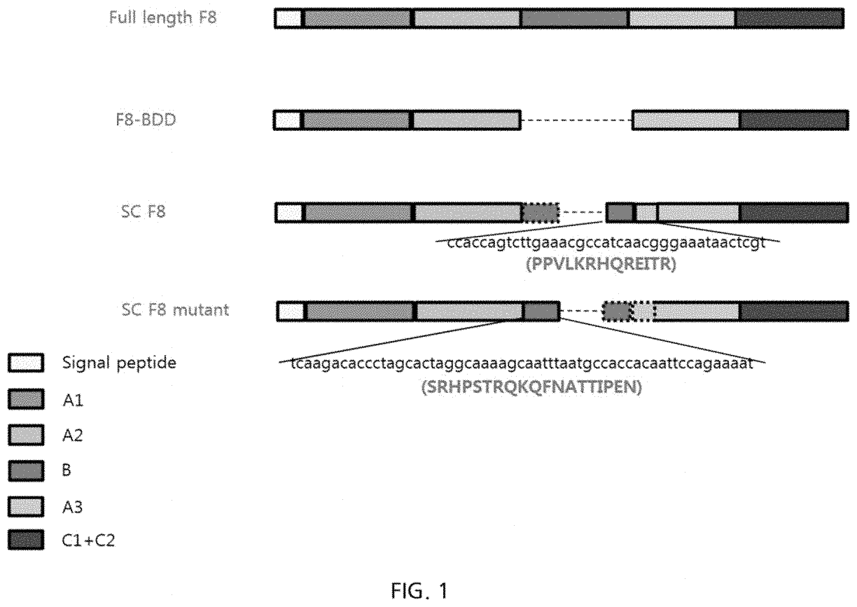

FIG. 2 shows the cleavage map of a pCDNA3.1 vector.

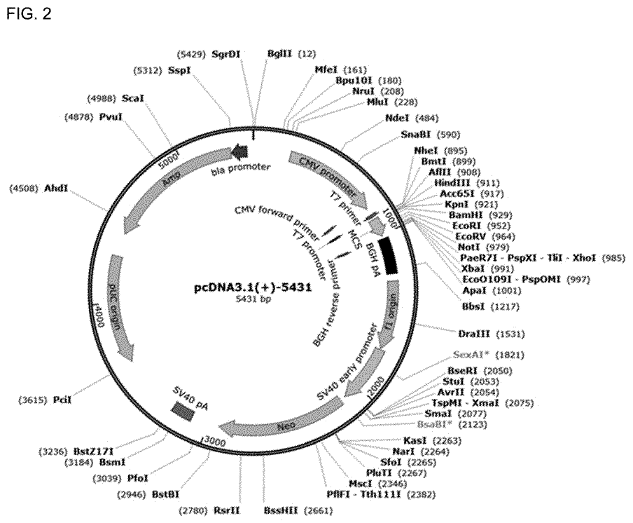

FIG. 3 shows the cleavage map of a pGP vector.

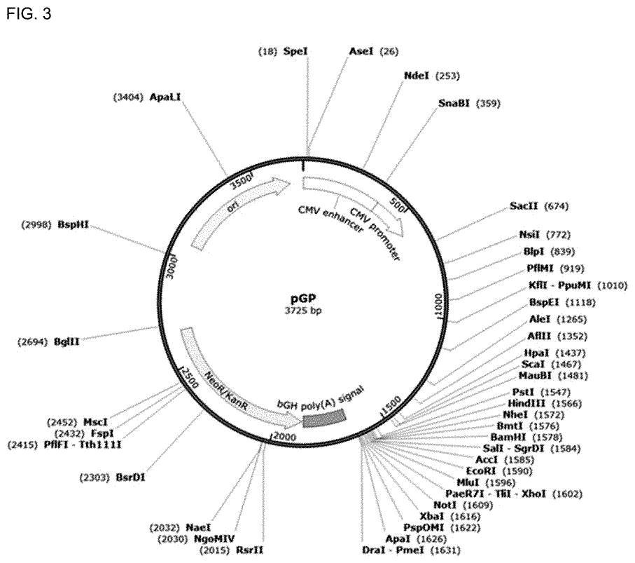

FIG. 4 shows the cleavage map of a pEF vector.

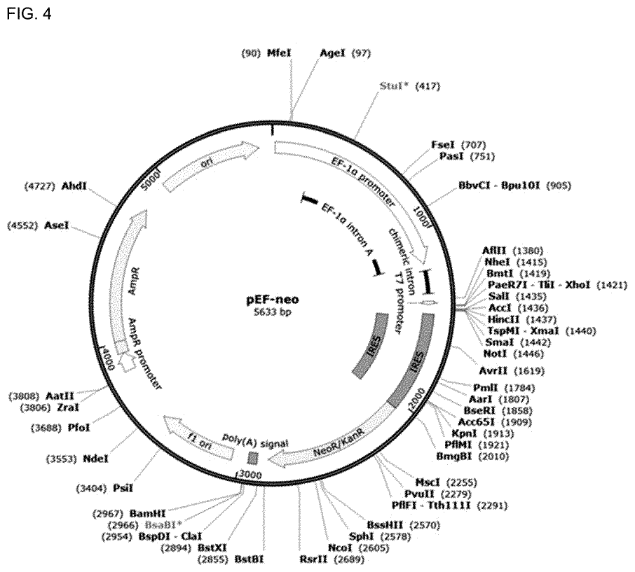

FIG. 5 shows a result of measuring protein expression level using a sample obtained from cells transfected with a pCDNA3.1 plasmid of Preparation Example 1.

FIG. 6 shows a result of measuring coagulant activity using a sample obtained from cells transfected with a pCDNA3.1 plasmid of Preparation Example 1.

FIG. 7 shows a result of measuring protein expression level using a sample obtained from cells transfected with a pGP plasmid of Preparation Example 2.

FIG. 8 shows a result of measuring protein expression level using a sample obtained from cells transfected with a pEP plasmid of Preparation Example 3.

### BEST MODE

Hereinafter, the present disclosure is described in detail.

In an aspect, the present disclosure provides a factor VIII mutant expression vector including a single-chain polynucleotide encoding a factor VIII mutant wherein amino acids Asp784 to Arg1671 are deleted from factor VIII represented by SEQ ID NO 1.

Factor VIII Mutant

In the present specification, the terms “coagulation factor VIII”, “factor VIII”, “FVIII” and “F8” are used interchangeably. Mature human factor VIII consists of 2351 amino acids (including a signal peptide) and has the following domains:

| | | | | | | | | |
|---|---|---|---|---|---|---|---|---|
| A1 | a1 | A2 | a2 | B | a3 | A3 | C1 | C2 |

signal peptide: residues 1-19,

A1: residues 20-355,

A2: residues 392-729,

B: residues 760-1667,

A3: residues 1709-2038,

C1: residues 2039-2191 and

C2: residues 2192-2351.

In addition, there are three acidic domains a1 (356-391), a2 (730-759) and a3 (1668-1708). The acidic domain a3 is known to be involved in binding of the factor VIII molecule to von Willebrand factor (vWF) which plays an important role in blood coagulation. During secretion, the factor VIII is cleaved between the B-domain and the a3 acidic domain, resulting in a heterodimer polypeptide. The factor VIII heterodimer is composed of a light chain (including A3, C1 and C2) and a heavy chain (including A1, A2 and B) of variable size. The heavy chain is heterologous due to limited proteolysis within the B-domain. In case of a heterodimer B-domain-deleted factor VIII, the “heavy chain” includes A1 and A2, but lacks a part or all of the B-domain.

The amino acid sequence of mature wild-type human coagulation factor VIII is shown in SEQ ID NO 1. The reference number to an amino acid position in a particular sequence refers to the location of the corresponding amino acid in the wild-type FVIII protein and does not exclude the presence of mutations (deletion, insertion and/or substitution) at other locations in the sequence. A DNA sequence encoding the SEQ ID NO 1 is SEQ ID NO 2.

The “coagulation factor VIII” includes not only the wild-type coagulation factor VIII but also a derivative of the wild-type coagulation factor VIII having the procoagulant activity of the wild-type coagulation factor VIII. The derivative may have deletion, insertion and/or addition as compared to the amino acid sequence of the wild-type factor VIII. A preferred derivative is an FVIII molecule from which all or a part of the B-domain has been deleted. The amino acid positions indicated throughout the present specification always refer to the positions of the respective amino acids in the mature full-length wild-type factor VIII (including a signal peptide cleavage).

In the present specification, the term “mutant” includes conservative or non-conservative substitution, insertion or deletion of an amino acid sequence, a nucleic acid sequence, etc. This variation does not substantially alter the active site or active domain which confers the biological activities of FVIII.

The factor VIII mutant according to the present disclosure refers to one wherein amino acids Asp784 to Arg1671 are deleted from factor VIII represented by SEQ ID NO 1. The mutant is a single-chain factor VIII mutant. The factor VIII mutant is one wherein a part of the B-domain (residues 784-1667) and a part of the a3 region (residues 1668-1671) are deleted, and the mutant has increased protein expression and improved coagulant activity and stability as compared to factor VIII, B-domain-deleted factor VIII and single-chain factor VIII. The factor VIII mutant has an amino acid sequence represented by SEQ ID NO 3.

The “single-chain factor VIII” refers to a factor VIII molecule which exists as a single polypeptide chain without being cleaved into two chains (e.g., a heavy chain and a light chain) during secretion from cells expressing the factor VIII molecule.

Polynucleotide

The present disclosure also relates to a polynucleotide encoding the factor VIII mutant having an amino acid sequence represented by SEQ ID NO 3.

In the present specification, the term “polynucleotide” refers to any polyribonucleotide or polydeoxyribonucleotide which may be an unmodified RNA or DNA, or a modified RNA or DNA. The polynucleotide of the present disclosure may be a single-chain DNA or RNA. The term “polynucleotide” used in the present specification includes a DNA or RNA containing a modified base and/or an unusual base, e.g., inosine. It is obvious that various modifications that provide known useful purposes can be made to the DNA or RNA. The term “polynucleotide” used in the present specification also includes a chemically, enzymatically or metabolically modified polynucleotide.

Those skilled in the art will understand that the factor VIII mutant may be encoded by several polynucleotides due to the degeneracy of the genetic code. That is to say, it is understood that a polynucleotide sequence encoding the factor VIII mutant that can be used in the present disclosure also includes a nucleotide sequence that exhibits substantial identity to the amino acid sequence represented by SEQ ID NO 3.

Specifically, the polynucleotide of the present disclosure may be an isolated polynucleotide. The “isolated” polynucleotide means a polynucleotide that is substantially free of other nucleic acid sequences, such as chromosomal and extrachromosomal DNA and RNA, although not being limited thereto. The isolated polynucleotide may be purified from a host cell. Common nucleic acid purification methods known to those skilled in the art may be used obtain the isolated polynucleotide. The term also includes a recombinant polynucleotide and a chemically synthesized polynucleotide.

Expression System for Expressing a Factor VIII Mutant

Expression Vector

In another aspect, the present disclosure provides an expression vector including a single-chain polynucleotide encoding the factor VIII mutant (hereinafter, referred to as a ‘factor VIII mutant expression vector’).

In the present specification, the term “expression” refers to production of the factor VIII mutant in a cell.

In the present specification, the term “expression vector” refers to a vector capable of expressing the factor VIII mutant in a suitable host cell, and refers to a gene construct containing an essential regulatory element to which a gene insert is operably linked in such a manner as to be expressed.

In the present specification, the term “operably linked” refers to functional linkage between a nucleic acid expression control sequence and a polynucleotide encoding the factor VIII mutant to perform a general function. For example, a promoter may be operably linked to a polynucleotide encoding the factor VIII mutant to affect the expression of the polynucleotide. The operable linkage with a recombinant vector may be achieved using a gene recombination technique well known in the art. For site-specific DNA cleavage and linkage, enzymes generally known in the art may be used.

The expression vector of the present disclosure is constructed using a plasmid, a vector or a viral vector, although not being limited thereto. A suitable expression vector may include a regulatory element such as a promoter, an operator, a start codon, a stop codon, a polyadenylation signal and an enhancer, and may be prepared in various manners depending on the intended use. The promoter of the vector may be constitutive or inducible.

Specifically, the expression vector may be prepared by using a vector selected from a group consisting of pCDNA3.1, pGP and pEF in terms of effect. Specifically, the factor VIII mutant expression vector has remarkably increased protein expression as compared to a polynucleotide encoding factor VIII represented by SEQ ID NO 1, B-domain-deleted factor VIII or single-chain factor VIII. In particular, it was confirmed that protein expression and stability can be further improved by using an endogenous promoter such as an EF1a promoter, etc. and by codon-optimizing (CO) the single-chain F8-BDD mutant.

In order to produce the factor VIII mutant at a high level in a host cell, an appropriate regulatory element is necessary in a recombinant expression vector that can be proliferated in various expression systems according to methods known to those skilled in the art, together with an assembly of modified cDNA. Effective transcriptional control elements may be derived from viruses having animal cells as natural hosts or from chromosomal DNA of animal cells. Specifically, a promoter-enhancer combination derived from the long terminal repeat of simian virus 40, adenovirus, BK polyomavirus, human cytomegalovirus or Rous sarcoma virus, or a promoter-enhancer combination including a strongly constitutively transcribed gene in animal cells, such as beta-actin or GRP78, may be used. In order to achieve stable, high levels of mRNA transcribed from cDNA, the transcriptional unit should contain in its 3′-proximal part a DNA region encoding a transcriptional termination-polyadenylation sequence. Specifically, this sequence is derived from the simian virus 40 early transcriptional region, rabbit beta-globin gene or human tissue plasminogen activator gene.

Expression of Factor VIII Mutant

The expression vector may be transfected into a suitable host cell and, as a result, may lead to the expression of the factor VIII mutant of the present disclosure and the production of a functional protein.

In another aspect, the present disclosure provides a host cell including the polynucleotide or the expression vector.

The host cell of the present disclosure may be used in a method for producing the factor VIII mutant of the present disclosure. The method may include: (a) a step of culturing the host cell under a condition where the factor VIII mutant can be expressed; and (b) a step of recovering the factor VIII mutant from the host cell or a culture medium.

Specifically, for expression of the factor VIII mutant, a nucleotide such as cDNA is incorporated into the genome of a suitable host cell. Specifically, the host cell should be an animal cell of vertebrate origin in order to ensure correct folding, disulfide bond formation, asparagine-linked glycosylation and other post-translational modifications and to ensure secretion into a culture medium. Examples of other post-translational modifications include 0-sulfation of tyrosine and proteolytic processing of a nascent polypeptide chain. Examples of the cell that can be used include monkey COS cells, mouse L cells, mouse C127 cells, hamster BHK-21 cells, human embryonic kidney 293 cells, and hamster CHO cells.

A recombinant expression vector encoding the corresponding cDNA may be introduced into an animal cell in different ways. For example, the recombinant expression vector can be constructed from vectors based on viruses. Examples include the vectors based on baculovirus, vaccinia virus and adenovirus, specifically bovine papillomavirus.

The transcriptional unit encoding the corresponding DNA can also be introduced into an animal cell together with another recombinant gene which may function as a dominant selection marker in the cell in order to facilitate the isolation of specific cell clones integrated to the recombinant DNA in the genome. Examples of the dominant selection marker gene include Tn5 aminoglycoside phosphotransferase conferring resistance to geneticin (G418), hygromycin phosphotransferase conferring resistance to hygromycin, and puromycin acetyltransferase conferring resistance to puromycin. The recombinant expression vector encoding such a selection marker may be present on the same vector as the vector encoding the cDNA of the desired protein, or may be encoded on a separate vector which is simultaneously introduced and integrated into the genome of the host cell, frequently resulting in a strong physical linkage between different transcriptional units.

Other types of the selection marker gene that can be used tougher with the cDNA of the desired protein are based on various transcriptional units encoding dihydrofolate reductase (DHFR). After introduction of this type of gene into cells lacking endogenous DHFR activity, typically CHO cells (DUKX-B11, DG-44), it will enable the cells to grow in a medium lacking nucleosides. For example, the medium may be Ham's F12 without hypoxanthine, thymidine and glycine. The DHFR gene can be introduced together with the factor VIII cDNA transcriptional unit into CHO cells, being linked on the same vector or on different vectors, thus creating DHFR-positive cells producing a recombinant protein.

If the cells are proliferated in the presence of the cytotoxic DHFR inhibitor methotrexate, new cells resistant to methotrexate will be produced. These cells can produce a recombinant protein at an increased rate due to the amplified number of transcriptional units linked to DHFR. When these cells are proliferated while increasing the concentration of methotrexate (from 1 to 10000 nM), new cells producing the desired protein can be obtained with very high rate.

The cells producing the desired protein can be proliferated on a large scale, through suspension culture or on various solid supports. Examples of the support include a microcarrier based on dextran or collagen matrix, or a solid support in the form of a hollow fiber or various ceramic materials. When proliferated through suspension culture or on a microcarrier, the culturing of the cells can be performed by bath culture or perfusion culture whereby conditioned media are produced continuously over extended periods of time. Thus, according to the present disclosure, the above-described cells are well suited for the development of an industrial process for the production of the desired recombinant mutant protein.

Pharmaceutical Composition

In addition, the present disclosure provides a pharmaceutical composition for preventing or treating hemorrhagic disease or hemorrhage, which contains the expression vector described above.

The hemorrhagic disease may be hemophilia A, hemophilia caused or complicated by an inhibitory antibody against factor VIII or factor Villa, or hemophilia B. In addition, the hemorrhagic disease may be one selected from a group consisting of neonatal coagulopathy, severe liver disease, thrombocytopenia, congenital deficiency of factor V, VII, X or XI, and von Willebrand disease with inhibitors against von Willebrand factor.

And, the hemorrhage may be caused by blood loss associated with a high-risk surgical procedure, traumatic blood loss, bone marrow transplantation or cerebral hemorrhage.

The pharmaceutical composition of the present disclosure may be for gene therapy.

Purification of Factor VIII Mutant

The factor VIII mutant which is secreted from the above-described cells and accumulated in the culture thereof can be concentrated and purified by a variety of biochemical and chromatographic methods, including methods utilizing differences in size, charge, hydrophobicity, solubility, specific affinity, etc. between the desired protein and other substances in the cell culture medium.

Specifically, the factor VIII mutant of the present disclosure may be purified to a purity of 80% or higher, more specifically 95% or higher. In particular, it is desired that the mutant is in a pharmaceutically pure state, with a purity higher than 99.9% with respect to contaminating macromolecules, particularly other proteins and nucleic acids, and free of infectious and pyrogenic agents. Specifically, the isolated or purified mutant of the present disclosure is substantially free of other unrelated polypeptides.

As an example of such purification, after adsorbing the factor VIII mutant onto a solid support, followed by washing and desorption, the protein may be further purified by various chromatographic techniques based on its properties. The sequence of purification steps is selected according to the ability or selectivity of the steps, the stability of the support, or other aspects. Preferred purification steps include, for example, ion-exchange chromatography, immunoaffinity chromatography, affinity chromatography, hydrophobic interaction chromatography, dye chromatography and size exclusion chromatography steps, although not being limited thereto.

To minimize the theoretical risk of viral contamination, additional steps may be included in the method to effectively inactivate or eliminate viruses. Such steps are, for example, heat treatment in liquid or solid state, solvent and/or surfactant treatment, light irradiation in the visible or UV spectrum, gamma irradiation or nanofiltration.

The modified polynucleotide (e.g., DNA) the present disclosure may also be incorporated into a transfer vector used in human gene therapy.

The various aspects described in the present specification may be combined with one another. Hereinafter, the present disclosure will be described in more detail by way of examples. The description of certain aspects of the present disclosure will be set forth in connection with the accompanying drawings.

Formulation

The inserted protein described in the present disclosure can be formulated into a pharmaceutical preparation for therapeutic use. The purified protein is dissolved in a common physiologically acceptable aqueous buffer solution and a pharmaceutical excipient may be optionally added thereto to provide a pharmaceutical preparation.

Pharmaceutical carriers and excipients as well as suitable pharmaceutical formulations are well known in the art (e.g., “Pharmaceutical Formulation of Peptides and Proteins”, Frokjaer et al., Taylor & Francis (2000) or “Handbook of Pharmaceutical Excipients”, 3rd edition, Kibbe et al., Pharmaceutical Press (2000)). In particular, a pharmaceutical composition containing the mutant of the present disclosure can be formulated into a lyophilized form or a stable liquid form. The mutant of the present disclosure can be lyophilized through a variety of procedures known in the art. The lyophilized formulation may be reconstituted prior to use by addition of one or more pharmaceutically acceptable diluent, such as injectable sterile water or sterile saline.

The formulation of the composition is delivered to a subject by any pharmaceutically appropriate means of administration. A variety of delivery systems are known and can be used to administer the composition by any convenient route. Typically, the composition of the present disclosure is administered systemically. In the case of systemic administration, the inserted protein of the present disclosure is formulated for parenteral (e.g., intravenous, subcutaneous, intramuscular, intraperitoneal, intracerebral, intrapulmonary, intranasal or transdermal) delivery or intestinal (e.g., oral, vaginal or rectal) delivery. The most preferred route of administration is intravenous and subcutaneous administration. These formulations may be administered continuously by infusion or bolus injection. Some formulations include sustained-release systems.

The inserted protein of the present disclosure may be administered to a patient in a therapeutically effective amount that does not reach a dose that causes unacceptable side effects and that is sufficient to produce the desired effect while preventing or reducing the severity or development of the condition or symptom to be treated. The exact dosage depends on a variety of factors, including signs, formulations and administration method, and each indication should be determined through preclinical and clinical trials.

The pharmaceutical composition of the present disclosure may be administered alone or in combination with other therapeutic agents. Such agents may be included in the same formulation. An example of such a formulation is von Willebrand factor.

Therapeutic Method

The present disclosure also relates to a method for treating a subject suffering from hemophilia A, hemophilia B or a hemorrhagic disease such as acquired hemophilia. The therapeutic method may include a step of administering an effective amount of a pharmaceutical composition containing the expression vector of the present disclosure to a subject in need thereof. Alternatively, it may include a step of administering an effective amount of the host cell of the present disclosure to the subject.

According to an exemplary embodiment of the present disclosure, the factor VIII mutant of the present disclosure may be administered in an amount of 10 ng to 100 mg, and a polynucleotide encoding the protein may be administered in an amount of 1 μg to 100 mg. When the factor VIII mutant or the polynucleotide encoding the same is administered more than once, each administration dosage may be the same or different.

Hereinafter, the present disclosure will be described in detail through examples. However, the following examples are for illustrative purposes only and the scope of the present disclosure is not limited by the examples.

### EXAMPLES

### Example 1. Construction of Factor VIII Mutant (F8M)

A fragment 1 represented by SEQ ID NO 5 was prepared through polymerase chain reaction (PCR) by using a full-length factor VIII gene represented by SEQ ID NO 1 as a template and using primers 1 and 2. The PCR was conducted by preparing a mixture solution of 2 μL of the template DNA, 1 μL of each primer at 10 pmol/μL, 2.5 μL of 2.5 mM dNTP, 1 μL of a Pfu enzyme mix (Enzynomics, Korea) and 2.5 μL of a 10× buffer by adding sterilized triply distilled water to 50 μL and repeating 40 cycles of 30 seconds at 95° C., 30 seconds at 60° C. and 30 seconds at 72° C. A fragment 2 represented by SEQ ID NO 6 was prepared in the same manner through PCR by using primers 8 and 3. Then, a single-chain factor VIII mutant was prepared by conducing overlapping PCR with the fragments 1 and 2 using primers 9 and 10. The overlapping PCR was conducted by in the same manner as described above except for adding 2 μL of the DNA fragment.

### Example 2. Construction of Factor VIII Mutant CO (F8M CO)

After establishing a codon-optimized base sequence on the basis of the base sequence of the factor VIII mutant prepared in Example 1 using the OptimumGene™ algorithm, a factor VIII mutant CO represented by SEQ ID NO 4 was synthesized by Genscript on the basis of the base sequence.

### Comparative Example 1. Construction of Factor VIII (F8)

A full-length factor VIII (F8) gene represented by SEQ ID NO 1 (NM_000132.3) was purchased from Origene Technologies (MD, USA).

### Comparative Example 2. Construction of B-Domain-Deleted Factor VIII (F8 BDD)

A fragment 3 represented by SEQ ID NO 7 was prepared through polymerase chain reaction (PCR) by using a full-length factor VIII gene represented by SEQ ID NO 1 as a template and using primers 1 and 4. The PCR was conducted by preparing a mixture solution of 2 μL of the template DNA, 1 μL of each primer at 10 pmol/μL, 2.5 μL of 2.5 mM dNTP, 1 μL of a Pfu enzyme mix (Enzynomics, Korea) and 2.5 μL of a 10× buffer by adding sterilized triply distilled water to 50 μL and repeating 40 cycles of 30 seconds at 95° C., 30 seconds at 60° C. and 30 seconds at 72° C. A fragment 4 represented by SEQ ID NO 8 was prepared in the same manner through PCR by using primers 8 and 5. Then, a B-domain-deleted factor VIII (F8 BDD) was prepared by conducing overlapping PCR with the fragments 3 and 4 using primers 9 and 10. The overlapping PCR was conducted by in the same manner as described above except for adding 2 μL of the DNA fragment.

### Comparative Example 3. Construction of Single-Chain Factor VIII (Sc F8)

A fragment 5 represented by SEQ ID NO 9 was prepared through polymerase chain reaction (PCR) by using a full-length factor VIII gene represented by SEQ ID NO 1 as a template and using primers 1 and 6. The PCR was conducted by preparing a mixture solution of 2 μL of the template DNA, 1 μL of each primer at 10 pmol/μL, 2.5 μL of 2.5 mM dNTP, 1 μL of a Pfu enzyme mix (Enzynomics, Korea) and 2.5 μL of a 10× buffer by adding sterilized triply distilled water to 50 μL and repeating 40 cycles of 30 seconds at 95° C., 30 seconds at 60° C. and 30 seconds at 72° C. A fragment 6 represented by SEQ ID NO 10 was prepared in the same manner through PCR by using primers 8 and 7. Then, a single-chain factor VIII mutant (sc F8) was prepared by conducing overlapping PCR with the fragments 5 and 6 using primers 9 and 10. The overlapping PCR was conducted by in the same manner as described above except for adding 2 μL of the DNA fragment.

The primers used in Examples and Comparative Examples are described in Table 1.

TABLE 1

| Primer | Primer | |
|---|---|---|
| No. | name | Base sequence |
| 1 | F8 (F) | ATGCAAATAGAGCTCTCCACCTGCTTCTTT |
| 2 | F8M-1 | TCTGACTGAAGAGTAGTATTTTCTGGAATT |
| | (R) | GTGGTGGCATTAAAT |
| 3 | F8M-2 | CCACAATTCCAGAAAATACTACTCTTCAGT |
| | (F) | CAGATCAAGAGGAAATTGAC |
| 4 | F8 BDD- | AGAGTAGTACGAGTTATTTCTCTTGGTTCA |
| | 1 (R) | ATGGCAT |
| 5 | F8 BDD- | GAACCAAGAGAAATAACTCGTACTACTCTT |
| | 2 (F) | CAGTCAGA |
| 6 | F8 Sc-1 | TCAAGACTGGTGGATTCTGGGAGAAGCTTC |
| | (R) | TTGGTTC |
| 7 | F8 Sc-2 | CTTCTCCCAGAATCCACCAGTCTTGAAACG |
| | (F) | CCA |
| 8 | F8 (R) | TCAGTAGAGGTCCTGTGCCTCGCAGCCCAG |
| 9 | F8 | GCTAGCATGCAAATAGAGCTCTCCACCTGC |
| | final | |
| | (F) | |
| 10 | F8 | GCGGCCGCTCAGTAGAGGTCCTGTGCCTCG |
| | final | CA |
| | (R) | |
| 11 | pGP (F) | GACGAATTCACGCGTCTCGAGGCGGCCGCT |
| | | CTAGAGGGCCCGTTTAAA |
| 12 | pGP (R) | GACGAATTCGTCGACGGATCCGCTAGCAAG |
| | | CTTCGTGTCAAGGACGGT |

### PREPARATION EXAMPLES

### Preparation Example 1. Construction of Expression Vector (pCDNA3.1)

Each of pCDNA3.1 (represented by SEQ ID NO 12) purchased from Genscript and the proteins prepared in Examples and Comparative Examples was cleaved with NheI and NotI enzymes for 1 hour and then fragments were separated by electrophoresing on agarose gel. The separated fragments were ligated for 30 minutes using T4 ligase, and then introduced into E. coli. After culturing the E. coli overnight, DNA was isolated from the cultured colony on the next day through mini-preparation and then investigated using NheI and NotI.

### Preparation Example 2. Construction of Expression Vector (pGP)

After synthesizing a pCK vector by referring to Lee, et al. (Lee Y, et al. Improved expression of vascular endothelial growth factor by naked DNA in mouse skeletal muscles: implication for gene therapy of ischemic diseases. Biochem. Biophys. Res. Commun. 2002; 272(1): 230-235), fragments were obtained by conducting PCR as described above using primers 11 and 12 described in Table 1. Using an EcoRI enzyme, the fragments were reacted at 37° C. for 1 hour, and then DNA was purified using an Expin Gel SV kit (GeneAll, Korea). Then, after ligating for 30 minutes using T4 ligase and introducing into E. coli, and then culturing the E. coli overnight, a pGP vector represented by SEQ ID NO 13 was constructed by isolating DNA from the cultured colony on the next day through mini-preparation.

Each of the prepared pGP vector and the fragments prepared in Examples and Comparative Examples was cleaved with NheI and NotI enzymes for 1 hour and then fragments were separated by electrophoresing on agarose gel. The separated fragments were ligated for 30 minutes using T4 ligase, and then introduced into E. coli. After culturing the E. coli overnight, DNA was isolated from the cultured colony on the next day through mini-preparation and then investigated using NheI and NotI.

### Preparation Example 3. Construction of Expression Vector (pEF)

Each of a pEF vector (represented by SEQ ID NO 14) purchased from ThermoFisher Scientifics and the DNA fragments prepared in Examples and Comparative Examples was cleaved with NheI and NotI enzymes for 1 hour and then fragments were separated by electrophoresing on agarose gel. The separated fragments were ligated for 30 minutes using T4 ligase, and then introduced into E. coli. After culturing the E. coli overnight, DNA was isolated from the cultured colony on the next day through mini-preparation and then investigated using NheI and NotI.

The prepared expression vectors are summarized in Table 2.

TABLE 2

| Expression vector | Expressed protein | Symbol | Vector |
|---|---|---|---|
| Control 1 | — | — | pCDNA3.1 |
| Example 1-1 | Factor VIII mutant | F8M | (Preparation |
| Example 2-1 | Codon-optimized factor | F8M CO | Example 1) |
| | VIII mutant | | |
| Comparative | Factor VIII | F8 | |
| Example 1-1 | | | |
| Comparative | B-domain-deleted factor | F8-BDD | |
| Example 2-1 | VIII | | |
| Comparative | Single-chain factor VIII | scF8 | |
| Example 3-1 | | | |
| Control 2 | — | — | pGP |
| Example 1-2 | Factor VIII mutant | F8M | (Preparation |
| Example 2-2 | Codon-optimized factor | F8M CO | Example 2) |
| | VIII mutant | | |
| Comparative | Factor VIII | F8 | |
| Example 1-2 | | | |
| Comparative | B-domain-deleted factor | F8-BDD | |
| Example 2-2 | VIII | | |
| Comparative | Single-chain factor VIII | scF8 | |
| Example 3-2 | | | |
| Control 3 | — | — | pEF |
| Example 1-3 | Factor VIII mutant | F8M | (Preparation |
| Example 2-3 | Codon-optimized factor | F8M CO | Example 3) |
| | VIII mutant | | |
| Comparative | Factor VIII | F8 | |
| Example 1-3 | | | |
| Comparative | B-domain-deleted factor | F8-BDD | |
| Example 2-3 | VIII | | |
| Comparative | Single-chain factor VIII | scF8 | |
| Example 3-3 | | | |

### TEST EXAMPLES

Preparation of Sample

The expression vectors prepared in Preparation Examples were purified according to a standard protocol (Qiagen). First, after transforming each plasmid DNA into E. coli, the E. coli was cultured overnight at 37° C. Centrifugation was conducted after lysing the E. coli cells with P1, P2 and P3. The obtained supernatant was passed through a maxi-prep column by gravity flow and a supernatant containing the plasmid DNA was obtained by using an elution buffer. After adding 0.7 volume equivalent of isopropanol, expression vectors were obtained by centrifuging at 12,000 rpm for 30 minutes using a high-speed centrifuge. Each expression vector was transfected into 5×10⁵ 293 T cells (ATCC CRL1573) using JetPEI (Polyplus, USA) according to the manufacturer's instructions. At 6 hours, the medium was replaced with a serum-free medium. Then, after culturing the cells at 37° C. and recovering supernatants on days 2 and 3, centrifugation was performed at 12,000 rpm for 5 minutes and the supernatant was stored at −80° C.

### Test Example 1. Protein Expression Level and Activity of Cells Including Expression Vector (pCDNA3.1)

Protein expression level and biological activity were measured for samples obtained from the cells transfected with the expression vectors of Preparation Example 1.

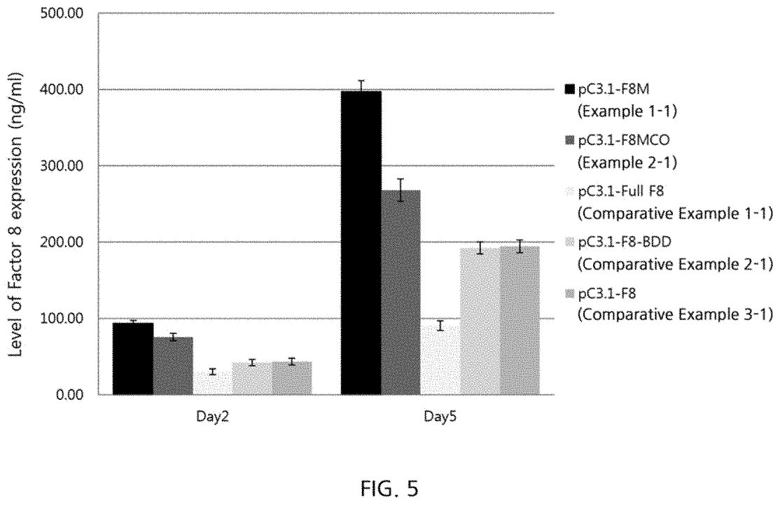

The protein expression level was measured using an ELISA kit (Stago Asserchrom VIII: Ag, France). First, the sample on day 2 was diluted to 1/5 and the sample on day 5 was diluted to 1/50. Then, 200 μL of a reference standard and the sample were added to a 96-well plate and incubated at 18-25° C. for 2 hours. After washing 5 times, the resultant was incubated with a secondary antibody at 18-25° C. for 2 hours. After washing 5 times and reacting with a TMB solution for 5 minutes, the reaction was stopped with a 1 M sulfuric acid solution and absorbance was measured at 450 nm using an ELISA reader. The result is shown in FIG. 5 and Table 3.

TABLE 3

| | Protein expression level (ng/mL) | |
|---|---|---|
| | Day 2 | Day 5 |
| Control 1 | — | — |
| Example 1-1 | 93.52 | 397.28 |
| Example 2-1 | 75.72 | 268.15 |
| Comparative Example 1-1 | 30.20 | 90.52 |
| Comparative Example 2-1 | 42.08 | 192.40 |
| Comparative Example 3-1 | 43.45 | 194.51 |

As seen from FIG. 5 and Table 3, the pCDNA3.1-F8 mutant (Example 1-1) showed significantly high protein expression level on both days 2 and 5. In particular, the difference in expression level was more distinct on day 5 than on day 2. In contrast, protein expression was not observed for the pCDNA3.1 vehicle (control 1).

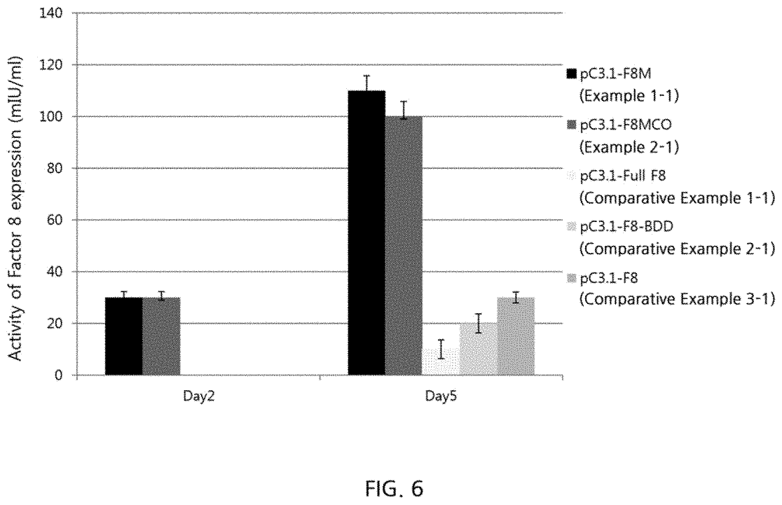

In addition, biological activity was measured using a coagulant activity assay kit (Stago, France). First, 1 IU/mL of a reference standard was serially diluted with an Owren-Koller buffer to obtain solutions with concentrations of 0.51, 0.25 and 0.13 IU/mL. Then, after adding 0.025 M CaCl₂, an APTT reagent activator and factor VIII-deficient plasma to the sample or the standard solution and incubating at 37° C., measurement was made using a coagulation analyzer (Stago Compact, France). The measurement result was normalized to the activity of the reference standard and the result is shown in FIG. 6 and Table 4.

TABLE 4

| | Coagulant activity (mIU/ml) | |
|---|---|---|
| | Day 2 | Day 5 |
| Control 1 | 0 | 0 |
| Example 1-1 | 30 | 110 |
| Example 2-1 | 30 | 100 |
| Comparative Example 1-1 | 0 | 0 |
| Comparative Example 2-1 | 0 | 20 |
| Comparative Example 3-1 | 0 | 30 |

As seen from FIG. 6 and Table 4, the CDNA3.1-F8 mutant (Example 1-1) showed remarkably high coagulant activity.

### Test Example 2. Protein Expression Level of Cells Including Expression Vector (PGP)

Protein expression level was measured for samples obtained from the cells transfected with the expression vectors of Preparation Example 2.

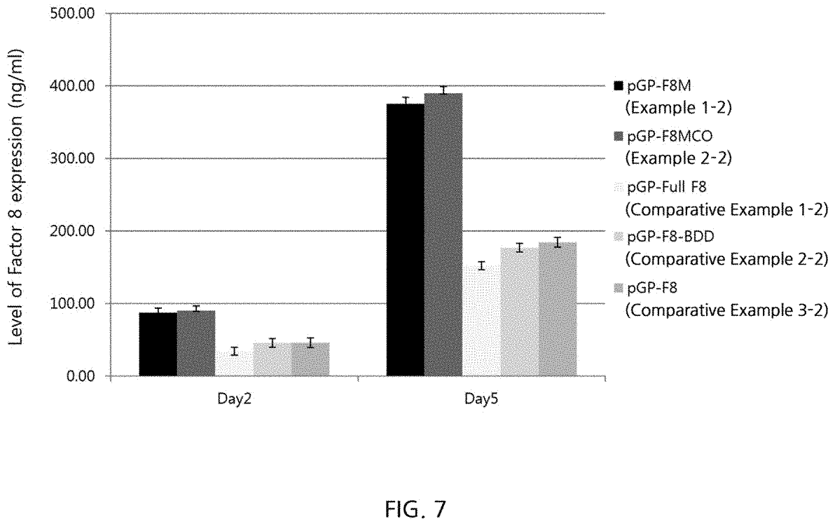

The protein expression level was measured using an ELISA kit (Stago Asserchrom VIII: Ag, France). First, the sample on day 2 was diluted to 1/5 and the sample on day 5 was diluted to 1/50. Then, 200 μL of a reference standard and the sample were added to a 96-well plate and incubated at 18-25° C. for 2 hours. After washing 5 times, the resultant was incubated with a secondary antibody at 18-25° C. for 2 hours. After washing 5 times and reacting with a TMB solution for 5 minutes, the reaction was stopped with a 1 M sulfuric acid solution and absorbance was measured at 450 nm using an ELISA reader. The result is shown in FIG. 7 and

Table 5.

TABLE 5

| | Protein expression level (ng/mL) | |
|---|---|---|
| | Day 2 | Day 5 |
| Control 2 | — | — |
| Example 1-2 | 44.76 | 179.05 |
| Example 2-2 | 67.44 | 269.67 |
| Comparative Example 1-2 | 21.13 | 76.25 |
| Comparative Example 2-2 | 24.60 | 94.80 |
| Comparative Example 3-2 | 31.00 | 120.90 |

As seen from FIG. 7 and Table 5, the pGP-F8 mutant (Example 1-2) showed significantly high protein expression level on both days 2 and 5. In particular, the difference in expression level was more distinct on day 5 than on day 2. In contrast, protein expression was not observed for the pGP vehicle (control 2).

### Test Example 3 Protein Expression Level of Cells Including Expression Vector (PEF)

Protein expression level was measured for samples obtained from the cells transfected with the expression vectors of Preparation Example 3.

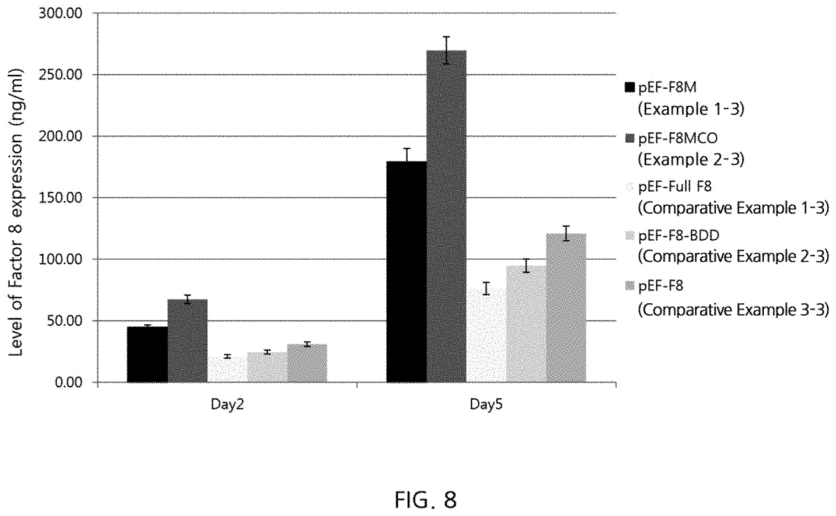

The protein expression level was measured using an ELISA kit (Stago Asserchrom VIII: Ag, France). First, the sample on day 2 was diluted to 1/5 and the sample on day 5 was diluted to 1/50. Then, 200 μL of a reference standard and the sample were added to a 96-well plate and incubated at 18-25° C. for 2 hours. After washing 5 times, the resultant was incubated with a secondary antibody at 18-25° C. for 2 hours. After washing 5 times and reacting with a TMB solution for 5 minutes, the reaction was stopped with a 1 M sulfuric acid solution and absorbance was measured at 450 nm using an ELISA reader. The result is shown in FIG. 8 and Table 6.

TABLE 6

| | Protein expression level (ng/mL) | |
|---|---|---|
| | Day 2 | Day 5 |
| Control 3 | — | — |
| Example 1-3 | 87.28 | 375.30 |
| Example 2-3 | 90.22 | 389.74 |
| Comparative Example 1-3 | 34.24 | 152.08 |
| Comparative Example 2-3 | 45.70 | 176.96 |
| Comparative Example 3-3 | 46.04 | 184.36 |

As seen from FIG. 8 and Table 6, the pEF-F8 mutant (Example 1-3) showed significantly high protein expression level on both days 2 and 5. In particular, the difference in expression level was more distinct on day 5 than on day 2. In contrast, protein expression was not observed for the pEF vehicle (control 3).

From the above results, it was confirmed that the factor VIII mutant (F8M) may exhibit higher protein expression as compared to factor VIII (F8). In particular, it is thought that the factor VIII mutant (F8M) protein has improved stability as compared to the factor VIII (F8) because the protein expression level is higher on day 5 than on day 2. In addition, the coagulant activity on day 5 was higher (about 2.5 times) for the factor VIII mutant (F8M) as compared to factor VIII (F8). Through this, it can be seen that the factor VIII mutant (F8M) gene of the present disclosure therapy agent expresses proteins with high coagulant activity at higher levels. Additionally, it was observed that the factor VIII mutant (F8M) exhibits further improved protein expression level and stability on day 5 when codon-optimized with a pEF vector having an EF1 a promoter (FIG. 8).

While the exemplary embodiments have been shown and described, it will be understood by those skilled in the art that various changes in form and details may be made thereto without departing from the spirit and scope of this disclosure as defined by the appended claims.

1. A factor VIII mutant expression vector comprising a single-chain polynucleotide encoding a factor VIII mutant wherein amino acids Asp784 to Arg1671 are deleted from factor VIII represented by SEQ ID NO 1.

2. The factor VIII mutant expression vector according to claim 1, wherein the factor VIII mutant has an amino acid sequence represented by SEQ ID NO 3.

3. The factor VIII mutant expression vector according to claim 1, wherein the vector is a vector selected from a group consisting of pCDNA3.1, pGP and pEF.

4. The factor VIII mutant expression vector according to claim 1, which has increased protein expression as compared to an expression vector comprising a polynucleotide encoding factor VIII represented by SEQ ID NO 1, B-domain-deleted factor VIII or single-chain factor VIII.

5. An expression system for expressing a factor VIII mutant, comprising the expression vector according to claim 1.

6. A pharmaceutical composition for preventing or treating hemorrhagic disease or hemorrhage, comprising the expression vector according to claim 1.

7. The pharmaceutical composition for preventing or treating hemorrhagic disease or hemorrhage according to claim 6, wherein the hemorrhagic disease is hemophilia A, hemophilia caused or complicated by an inhibitory antibody against factor VIII or factor Villa, or hemophilia B.

8. The pharmaceutical composition for preventing or treating hemorrhagic disease or hemorrhage according to claim 6, wherein the hemorrhagic disease is one selected from a group consisting of neonatal coagulopathy, severe liver disease, thrombocytopenia, congenital deficiency of factor V, VII, X or XI, and von Willebrand disease with inhibitors against von Willebrand factor.

9. The pharmaceutical composition for preventing or treating hemorrhagic disease or hemorrhage according to claim 6, wherein the hemorrhage is caused by blood loss associated with a high-risk surgical procedure, traumatic blood loss, bone marrow transplantation or cerebral hemorrhage.

10. The pharmaceutical composition for preventing or treating hemorrhagic disease or hemorrhage according to claim 6, which is for gene therapy.


SEQUENCE LISTING (14 sequences)

SEQ ID NO: 1 - protein, 2351 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - DNA, 7056 bp, Homo sapiens
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 3 - protein, 1463 aa, Artificial Sequence
  factor 8 mutant
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 4 - protein, 4392 aa, Artificial Sequence
  factor 8 mutant CO
ATGCAGATTGAGCTGTCCACCTGTTTCTTCCTGTGCCTGCTGAGATTTTGCTTTTCCGCCACTCGCCGATACTACCTGGGGGCTGTGGAGCTGAGCTGGGATTACATGCAGTCCGACCTGGGAGAGCTGCCAGTGGACGCAAGGTTTCCACCTCGCGTGCCAAAGTCTTTCCCCTTTAATACAAGCGTGGTGTATAAGAAAACCCTGTTCGTGGAGTTTACAGATCACCTGTTCAACATCGCCAAGCCAAGGCCACCCTGGATGGGCCTGCTGGGACCAACCATCCAGGCAGAGGTGTACGACACCGTGGTCATCACCCTGAAGAACATGGCCTCTCACCCCGTGAGCCTGCACGCAGTGGGCGTGTCTTACTGGAAGGCCAGCGAGGGCGCCGAGTATGACGATCAGACCAGCCAGCGCGAGAAGGAGGACGATAAGGTGTTTCCTGGCGGCTCCCACACCTACGTGTGGCAGGTGCTGAAGGAGAATGGCCCTATGGCCAGCGACCCACTGTGCCTGACCTACTCTTATCTGAGCCACGTGGACCTGGTGAAGGATCTGAACTCCGGCCTGATCGGCGCCCTGCTGGTGTGCAGAGAGGGCTCTCTGGCCAAGGAGAAAACCCAGACCCTGCACAAGTTCATCCTGCTGTTCGCCGTGTTTGACGAGGGCAAGTCCTGGCACAGCGAGACAAAGAACAGCCTGATGCAGGACAGGGATGCAGCCTCTGCCCGCGCCTGGCCCAAGATGCACACAGTGAACGGCTACGTGAATAGGTCCCTGCCTGGCCTGATCGGATGCCACCGCAAGAGCGTGTACTGGCACGTGATCGGCATGGGCACCACACCTGAGGTGCACTCCATCTTCCTGGAGGGCCACACCTTTCTGGTGCGGAACCACAGACAGGCCAGCCTGGAGATCAGCCCAATCACCTTCCTGACAGCCCAGACCCTGCTGATGGATCTGGGCCAGTTCCTGCTGTTTTGCCACATCAGCTCCCACCAGCACGATGGCATGGAGGCCTACGTGAAGGTGGACAGCTGTCCTGAGGAGCCACAGCTGCGGATGAAGAACAATGAGGAGGCCGAGGACTATGACGATGACCTGACAGACTCCGAGATGGATGTGGTGCGGTTCGATGACGATAACTCTCCCAGCTTTATCCAGATCCGGAGCGTGGCCAAGAAGCACCCTAAGACCTGGGTGCACTACATCGCCGCCGAGGAGGAGGACTGGGATTATGCCCCCCTGGTGCTGGCCCCTGACGATAGAAGCTACAAGTCCCAGTATCTGAACAATGGCCCACAGAGGATCGGCCGCAAGTACAAGAAGGTGAGGTTCATGGCCTATACAGACGAGACATTCAAGACACGCGAGGCCATCCAGCACGAGAGCGGCATCCTGGGACCACTGCTGTACGGAGAAGTGGGCGACACCCTGCTGATCATCTTTAAGAACCAGGCCTCTAGGCCATACAATATCTATCCCCACGGCATCACAGATGTGCGCCCACTGTACAGCCGGAGACTGCCCAAGGGCGTGAAGCACCTGAAGGACTTCCCCATCCTGCCTGGCGAGATTTTCAAGTATAAGTGGACCGTGACAGTGGAGGATGGCCCTACCAAGTCCGACCCACGGTGCCTGACAAGATACTATTCTAGCTTCGTGAATATGGAGCGGGATCTGGCCAGCGGCCTGATCGGACCACTGCTGATCTGTTACAAGGAGAGCGTGGATCAGCGGGGCAACCAGATCATGTCCGACAAGAGAAATGTGATCCTGTTCAGCGTGTTTGACGAGAACCGGTCCTGGTATCTGACCGAGAACATCCAGAGATTCCTGCCAAATCCAGCAGGAGTGCAGCTGGAGGACCCCGAGTTTCAGGCCTCCAACATCATGCACTCTATCAATGGCTACGTGTTCGACAGCCTGCAGCTGAGCGTGTGCCTGCACGAGGTGGCCTACTGGTATATCCTGTCCATCGGCGCCCAGACCGATTTCCTGAGCGTGTTCTTCAGCGGCTACACCTTCAAGCACAAGATGGTGTATGAGGACACCCTGACACTGTTCCCATTTTCTGGCGAGACAGTGTTCATGAGCATGGAGAATCCCGGCCTGTGGATTCTGGGCTGCCACAACAGCGATTTCAGGAATCGCGGCATGACCGCCCTGCTGAAGGTGTCCTCTTGTGACAAGAACACAGGCGACTACTATGAGGATAGCTACGAGGACATCTCCGCCTATCTGCTGTCTAAGAACAATGCCATCGAGCCTAGGTCCTTTTCTCAGAACAGCCGGCACCCATCTACCAGACAGAAGCAGTTCAACGCCACCACAATCCCTGAGAATACCACACTGCAGTCTGATCAGGAGGAGATCGACTACGACGATACAATCAGCGTGGAGATGAAGAAGGAGGACTTCGACATCTACGACGAGGATGAGAACCAGTCTCCAAGGAGCTTCCAGAAGAAAACCCGGCACTACTTTATCGCCGCCGTGGAGCGCCTGTGGGATTATGGCATGAGCAGCAGCCCTCACGTGCTGCGGAATAGAGCCCAGAGCGGCTCCGTGCCACAGTTCAAGAAGGTGGTGTTCCAGGAGTTTACCGACGGCTCCTTTACACAGCCACTGTACAGGGGAGAGCTGAACGAGCACCTGGGCCTGCTGGGACCCTATATCAGAGCCGAGGTGGAGGATAACATCATGGTGACCTTCAGGAATCAGGCCAGCCGCCCCTACTCCTTTTATAGCTCCCTGATCAGCTACGAGGAGGACCAGAGGCAGGGAGCAGAGCCTAGAAAGAACTTCGTGAAGCCAAACGAGACAAAGACCTACTTTTGGAAGGTGCAGCACCACATGGCCCCTACCAAGGACGAGTTCGATTGCAAGGCCTGGGCCTATTTTTCTGACGTGGATCTGGAGAAGGACGTGCATTCCGGCCTGATCGGCCCTCTGCTGGTGTGCCACACCAACACACTGAATCCAGCCCACGGCCGGCAGGTCACCGTGCAGGAGTTCGCCCTGTTCTTTACCATCTTTGATGAGACAAAGTCCTGGTACTTCACAGAGAACATGGAGCGGAATTGCAGAGCCCCCTGTAATATCCAGATGGAGGACCCTACCTTCAAGGAGAACTACAGATTTCACGCCATCAATGGCTATATCATGGATACACTGCCAGGCCTGGTCATGGCACAGGACCAGCGGATCAGATGGTATCTGCTGAGCATGGGCAGCAACGAGAATATCCACTCTATCCACTTCAGCGGCCACGTGTTTACCGTGAGGAAGAAGGAGGAGTATAAGATGGCCCTGTACAACCTGTATCCCGGCGTGTTCGAGACAGTGGAGATGCTGCCTAGCAAGGCAGGCATCTGGAGGGTGGAGTGCCTGATCGGAGAGCACCTGCACGCAGGAATGTCCACCCTGTTTCTGGTGTACTCTAATAAGTGTCAGACACCACTGGGAATGGCCAGCGGACACATCAGGGATTTCCAGATCACCGCCTCCGGACAGTACGGACAGTGGGCACCTAAGCTGGCCCGCCTGCACTATTCTGGCAGCATCAACGCCTGGTCCACCAAGGAGCCTTTCTCTTGGATCAAGGTGGACCTGCTGGCCCCAATGATCATCCACGGCATCAAGACACAGGGCGCCAGACAGAAGTTCTCTAGCCTGTACATCAGCCAGTTTATCATCATGTATTCCCTGGATGGCAAGAAGTGGCAGACCTACAGAGGCAATAGCACCGGCACACTGATGGTGTTCTTTGGCAACGTGGACTCCTCTGGCATCAAGCACAACATCTTCAATCCTCCAATCATCGCCAGGTACATCCGCCTGCACCCAACCCACTATAGCATCAGGTCCACACTGCGCATGGAGCTGATGGGCTGCGATCTGAACTCCTGTTCTATGCCCCTGGGCATGGAGTCCAAGGCCATCTCTGACGCCCAGATCACCGCCAGCTCCTACTTCACCAATATGTTTGCAACATGGAGCCCCTCCAAGGCCCGGCTGCACCTGCAGGGCCGGTCCAACGCATGGCGGCCCCAGGTGAACAATCCTAAGGAGTGGCTGCAGGTGGATTTTCAGAAAACCATGAAGGTGACAGGCGTGACCACACAGGGCGTGAAGAGCCTGCTGACCTCCATGTACGTGAAGGAGTTCCTGATCTCTAGCTCCCAGGACGGCCACCAGTGGACACTGTTCTTTCAGAACGGCAAGGTGAAGGTGTTCCAGGGCAATCAGGATTCTTTTACCCCTGTGGTGAACAGCCTGGACCCCCCTCTGCTGACACGGTATCTGAGAATCCACCCACAGAGCTGGGTGCATCAGATTGCCCTGCGAATGGAAGTGCTGGGGTGTGAAGCTCAGGACCTGTATTGA

SEQ ID NO: 5 - DNA, 2366 bp, Artificial Sequence
  Fragment 1 for factor 8 mutant
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatactactcttcagtcaga

SEQ ID NO: 6 - DNA, 2060 bp, Artificial Sequence
  Fragment 2 for factor 8 mutant
ccacaattccagaaaatactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 7 - DNA, 2297 bp, Artificial Sequence
  Fragment 3 for BDD-factor 8
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagagaaataactcgtactactct

SEQ ID NO: 8 - DNA, 2064 bp, Artificial Sequence
  Fragment 4 for BDD-factor 8
gaaccaagagaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 9 - DNA, 2305 bp, Artificial Sequence
  Fragment 5 for sc factor 8
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaatccaccagtcttga

SEQ ID NO: 10 - DNA, 2095 bp, Artificial Sequence
  Fragment 6 for sc factor 8
cttctcccagaatccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 11 - DNA, 4374 bp, Artificial Sequence
  sc factor 8
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaatccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 12 - DNA, 5431 bp, Artificial Sequence
  pCDNA3.1
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 13 - DNA, 4374 bp, Artificial Sequence; identical to SEQ ID NO: 11
  pGP

SEQ ID NO: 14 - DNA, 5633 bp, Artificial Sequence
  pEF vector
ccaattcaagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttggcctttttgagtttggatcttggttcattctcaagcctcagacagtggttcaaagtttttttcttccatttcaggtgtcgtgaacacgggctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagatcctagagtcgacccgggcggccgctctagcccaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatgggcattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatccc